(19) United States
(12) Patent Application Publication
(10) Pub. No.: US 2022/0211430 A1
(43) Pub. Date: Jul. 7, 2022

(54) Medical Device

(71) Applicant: TERUMO KABUSHIKI KAISHA, Tokyo (JP)

(72) Inventor: Yusuke TAKAHASHI, Hadano-shi (JP)

(73) Assignee: TERUMO KABUSHIKI KAISHA, Tokyo (JP)

(21) Appl. No.: 17/704,347

(22) Filed: Mar. 25, 2022

Related U.S. Application Data
(63) Continuation of application No. PCT/JP2020/036816, filed on Sep. 29, 2020.

(30) Foreign Application Priority Data
Sep. 30, 2019 (JP) 2019-178331

(51) Int. Cl. A61B 18/14

(52) U.S. Cl. CPC A61B 18/1492; A61B 2017/00247

(57) ABSTRACT

A medical device is disclosed that can reduce variations in the degree of cauterization by an energy transfer element, and can suppress generation of thrombus formation, tissue damage, and the like due to the cauterization. The medical device can include an elongated shaft portion, and an expansion body that is provided in a distal portion of the shaft portion and can expand and contract in a radial direction, in which: the expansion body includes a plurality of wire portions that are linked with a shaft portion, and a clamping portion that is formed by at least one wire portion and clamps a biological tissue; the clamping portion includes an energy transfer element that outputs energy, and a back support portion; the back support portion includes a receiving surface that can face the energy transfer element when the expansion body expands; and the receiving surface can be inclined.

### CROSS-REFERENCES TO RELATED APPLICATIONS

This application is a continuation of International Application No. PCT/JP2020/036816 filed on Sep. 29, 2020, which claims priority to Japanese Application No. 2019-178331 filed on Sep. 30, 2019, the entire content of both of which is incorporated herein by reference.

### TECHNOLOGICAL FIELD

This disclosure relates to a medical device for applying energy to a biological tissue.

### BACKGROUND DISCUSSION

Chronic heart failure is a known heart disease. Chronic heart failure is broadly classified into a systolic heart failure and a diastolic heart failure, based on a cardiac function index. In a patient suffering from diastolic heart failure, myocardial hypertrophy appears, and stiffness (hardness) increases, so that blood pressure increases in a left atrium, and a cardiac pumping function is degraded. In this manner, the patient may show heart failure symptoms such as a pulmonary edema. In addition, another heart disease of the patient who shows the heart failure symptom because blood pressure increases on a right atrium side due to pulmonary hypertension, and the cardiac pumping function is degraded.

In recent years, shunt treatments have attracted attention in which, for the patients who suffer from heart failure, a shunt (through-hole) serving as an escape route for increased atrial pressure is formed in an atrial septum, thereby enabling heart failure symptoms to be alleviated. In the shunt treatment, the atrial septum is accessed using an intravenous approaching method, and the through-hole is formed to have a desired size. For example, a medical device disclosed in U.S. Pat. No. 8,882,697 is used as one of medical devices for performing the shunt treatment on the atrial septum.

In the medical device of U.S. Pat. No. 8,882,697, a shunt hole is enlarged using a balloon serving as an expansion body that is provided in a distal portion of a shaft portion, and the shunt hole is maintained by an electrode provided in the balloon. In this medical device, the electrode (energy transfer element) is exposed to blood, so that energy is provided into the blood and an unintended site, which may cause variations in the degree of cauterization, formation of a thrombus, generation of tissue damage and the like.

### SUMMARY

A medical device is disclosed that can reduce variations in the degree of cauterization by an energy transfer element, and can suppress generation of thrombus formation, tissue damage, and the like due to the cauterization.

A medical device is disclosed that can include: an elongated shaft portion; and an expansion body that is provided in a distal portion of the shaft portion, and can expand and contract in a radial direction, in which the expansion body includes a plurality of wire portions that are linked with the shaft portion, and at least one clamping portion that is formed by at least one of the wire portions, the clamping portion includes an energy transfer element that outputs energy, and a back support portion, the back support portion includes a receiving surface that can face the energy transfer element when the expansion body expands, and the receiving surface is configured to be inclined so as to be approximately parallel to the energy transfer element, when the energy transfer element moves toward the back support portion.

With the medical device configured as the above, the receiving surface is approximately parallel to the energy transfer element in accordance with the movement of the energy transfer element, so that the energy transfer element can come into close contact with the biological tissue that is clamped between the energy transfer element and the receiving surface. Therefore, variations in the degree of cauterization by the energy transfer element can be reduced. Moreover, the energy transfer element can be prevented from locally floating from the biological tissue. Therefore, the energy transfer element can be prevented from supplying energy into blood and an unintended site, and generation of thrombus formation, tissue damage, and the like due to the cauterization can be suppressed.

The at least one clamping portion may include two outer peripheral portions on both sides in a width direction that is a direction orthogonal to an axis direction of the expansion body, and a direction orthogonal to the radial direction of the expansion body, relative to the back support portion. In this manner, the outer peripheral portions effectively guide the energy transfer element that moves toward the back support portion, to the back support portion that is positioned between the outer peripheral portions. Therefore, the energy transfer element can press the biological tissue supported by the two outer peripheral portions, and can press the biological tissue against the back support portion that is disposed between the two outer peripheral portions. Therefore, the energy transfer element comes into close contact with the biological tissue and is relatively hard to float from the biological tissue, and the position of the energy transfer element relative to the biological tissue is stably maintained by the two outer peripheral portions.

The two outer peripheral portions each may have a convex shape to an outer side in the width direction. In this manner, between the two outer peripheral portions, a wide region in which the back support portion is disposed can be secured. Moreover, the two outer peripheral portions in the width direction can support the biological tissue in the wide region, so that the energy transfer element and the receiving surface that clamp the biological tissue between the two outer peripheral portions can be rather easily maintained in the suitable positions.

The two outer peripheral portions each may have a circular arc shape that smoothly projects to the outer side in the width direction. In this manner, the outer peripheral portions is configured to be stored in an inner surface of a storage sheath without being caught on the inner surface of the storage sheath, which is a tubular member that stores the expansion body so as to be releasable, for example. Accordingly, the outer peripheral portions can be smoothly stored in the storage sheath, and can be smoothly released from the storage sheath.

Moreover, a maximum width of the outer peripheral portions that sandwich the back support portion between the two outer peripheral portions in the width direction may be larger than a maximum width of the energy transfer element in the width direction. In this manner, the outer peripheral portion easily guides a press direction of the energy transfer element so as to head for the back support portion.

The back support portion may move larger than the two outer peripheral portions due to a force in the axis direction to be received from the energy transfer element. In this manner, the back support portion can flexibly receive the biological tissue that is pressed by the energy transfer element while moving rearward larger in the press direction of the energy transfer element than the outer peripheral portion. Therefore, the energy transfer element comes into close contact with the biological tissue and is hard to float from the biological tissue.

The expansion body may include an inner projection portion between the energy transfer element and the back support portion, and the maximum width between the two outer peripheral portions that sandwich the back support portion between the two outer peripheral portions in the width direction may be larger than a maximum width of the inner projection portion in the width direction. In this manner, while maintaining the flexibility of the inner projection portion, a structure in which the width between the two outer peripheral portions is widened can be obtained.

The back support portion may have a cantilever beam shape that extends from the wire portion. In this manner, the back support portion can warp flexibly by receiving a force.

The back support portion may be a member that is supported by at least one flexible support wire that extends from the two outer peripheral portions that sandwich the back support portion between the two outer peripheral portions. In this manner, the back support portion can move lager than the outer peripheral portions due to a force in the axis direction.

The back support portion may be at least one flexible back support wire that extends from the two outer peripheral portions that sandwich the back support portion between the two outer peripheral portions. In this manner, the back support portion can warp more flexibly than the outer peripheral portions due to a force in the axis direction.

The back support portion may be a mesh-like member that extends from the two outer peripheral portions that sandwich the back support portion between the two outer peripheral portions. In this manner, the back support portion can warp more flexibly than the outer peripheral portions due to a force in the axis direction.

The back support portion may be a film body that extends from the two outer peripheral portions that sandwich the back support portion between the two outer peripheral portions. In this manner, the back support portion can warp more flexibly than the outer peripheral portions due to a force in the axis direction.

In accordance with an aspect, a medical device is disclosed comprising: an elongated shaft portion; and an expansion body that is provided in a distal portion of the shaft portion, and is configured to expand and contract in a radial direction, wherein the expansion body includes a plurality of wire portions that are linked with the shaft portion, and at least one clamping portion that is formed by at least one of the wire portions, the clamping portion includes an energy transfer element configured to output energy, and a back support portion, the at least one clamping portion includes two outer peripheral portions on both sides in a width direction, the back support portion includes at least one flexible back support wire that extends from the two outer peripheral portions that sandwich the back support portion between the two outer peripheral portions, and the receiving surface is configured to be inclined to be approximately parallel to the energy transfer element, when the energy transfer element moves toward the back support portion.

In accordance with another aspect, a method for treatment, the method comprising: expanding a through-hole formed in an atrial septum to allow a right atrium and a left atrium of a heart failure patient to communicate with each other; confirming hemodynamics of blood flow in a vicinity of the through-hole; performing maintenance treatment for maintaining a size of the through-hole with a medical device comprising an elongated shaft portion, and an expansion body that is provided in a distal portion of the shaft portion, the expansion body configured to expand and contract in a radial direction, wherein the expansion body includes a plurality of wire portions that are linked with the shaft portion, and at least one clamping portion that is formed by at least one of the wire portions, the clamping portion includes an energy transfer element configured to output energy, and a back support portion, and wherein the back support portion includes a receiving surface; expanding the expansion body in the through hole so that the receiving surface faces the energy transfer element; and moving the energy transfer element toward the back support portion so that the receiving surface is approximately parallel to the energy transfer element.

### BRIEF DESCRIPTION OF THE DRAWINGS

FIG. 1 is a front view illustrating an overall configuration of a medical device according to an embodiment.

FIG. 2 is an enlarged perspective view illustrating the vicinity of an expansion body.

FIG. 3 is a front view illustrating a state where one of wire portions is extended to be flat.

FIG. 4 is a view for describing a treatment method using the medical device according to the present embodiment, and is a view for schematically describing a state where the expansion body is disposed in a through-hole of an atrial septum, in which the medical device is illustrated in a front view and a biological tissue is illustrated in a cross-sectional view, respectively.

FIG. 5 is a view for schematically describing a state where a distal portion of the medical device is inserted into the atrial septum, in which a part of the medical device is illustrated in a front view, and a storage sheath and the biological tissue are illustrated in a cross-sectional view, respectively.

FIG. 6 is a view for schematically describing a state where a site on a distal side of the expansion body is developed on a left atrium side, in which the medical device is illustrated in a front view and the biological tissue is illustrated in a cross-sectional view, respectively.

FIG. 7 is a view for schematically describing a state where the expansion body is disposed in the atrial septum, in which the medical device is illustrated in a front view and the biological tissue is illustrated in a cross-sectional view, respectively.

FIG. 8 is a view for schematically describing a state where a diameter of the expansion body is increased in the atrial septum, in which the medical device is illustrated in a front view and a biological tissue is illustrated in a cross-sectional view, respectively.

FIGS. 9A and 9B are cross-sectional views illustrating the atrial septum and the expansion body, and wherein FIG. 9A illustrates a state before the atrial septum is clamped by a distal side clamping portion and a proximal side clamping portion, and FIG. 9B illustrates a state where the atrial septum is clamped by the distal side clamping portion and the proximal side clamping portion.

FIGS. 10A and 10B are cross-sectional views illustrating the atrial septum and the expansion body, and wherein FIG. 10A is a cross-sectional view taken along XA-XA line in FIG. 9A, and FIG. 10B is a cross-sectional view taken along XB-XB line in FIG. 9B.

FIG. 11 is a view for schematically describing a state where the through-hole of the atrial septum is enlarged by the expansion body, in which the medical device is illustrated in a front view and a biological tissue is illustrated in a cross-sectional view, respectively.

FIG. 12 is an enlarged perspective view illustrating the vicinity of an expansion body of a medical device according to a first modification example.

FIGS. 13A to 13D are enlarged perspective views each illustrating the vicinity of a distal side clamping portion of a medical device according to modification examples, and wherein FIG. 13A illustrates a second modification example, FIG. 13B illustrates a third modification example, FIG. 13C illustrates a fourth modification example, and FIG. 13D illustrates a fifth modification example.

### DETAILED DESCRIPTION

Set forth below with reference to the accompanying drawings is a detailed description of embodiments of a medical device for applying energy to a biological tissue representing examples of the inventive medical device for applying energy to a biological tissue. The size ratios on the drawings may be exaggerated or different from the actual ratios for convenience of description and illustration. Moreover, in the present specification, a side of a medical device 10 to be inserted into a lumen of a living body is referred to as a “distal side”, and a side at which the medical device 10 is operated is referred to as a “proximal side”.

The medical device 10 according to the present embodiment is configured, as shown in FIG. 4, such that a through-hole Hh formed in an atrial septum HA of a heart H of a patient can be enlarged, and further, a maintenance treatment can be performed that maintains the through-hole Hh having been enlarged to the increased sized.

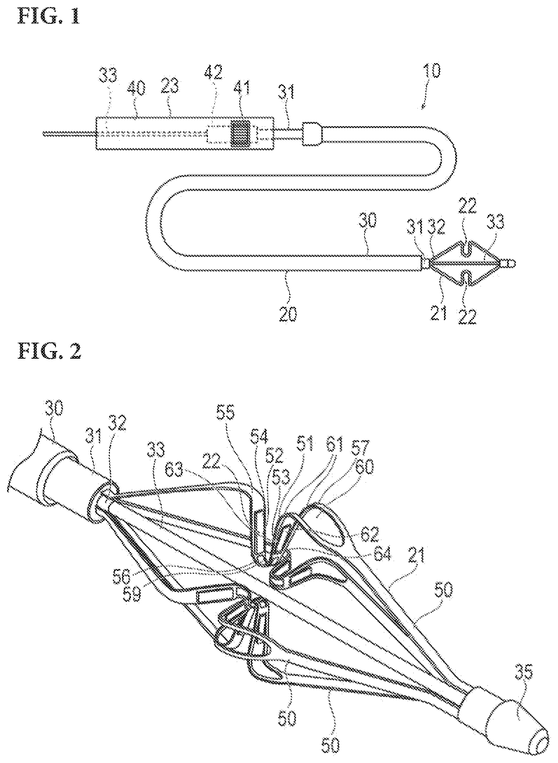

As shown in FIG. 1, the medical device 10 according to the present embodiment includes an elongated shaft portion 20, an expansion body 21 that is provided in a distal portion of the shaft portion 20, and an operation unit 23 that is provided in a proximal portion of the shaft portion 20. In the expansion body 21, an energy transfer element 22 for performing the aforementioned maintenance treatment is provided.

The shaft portion 20 includes an outer shaft 31 that holds the expansion body 21 in a distal portion of the outer shaft 31, and a storage sheath 30 that stores the outer shaft 31. The storage sheath 30 is movable forward and rearward in an axis direction (or axial direction) relative to the outer shaft 31. The storage sheath 30 in a state of having moved to a distal side of the shaft portion 20 can store the expansion body 21 in an inside of the storage sheath 30. The storage sheath 30 is moved to a proximal side in a state where the expansion body 21 is stored to enable the expansion body 21 to be exposed.

A pulling shaft 33 is stored in an inside of the outer shaft 31. The pulling shaft 33 protrudes from a distal end of the outer shaft 31 to the distal side, and has a distal portion that is fixed to a distal member 35. A proximal portion of the pulling shaft 33 extends to the proximal side of the operation unit 23. The distal member 35 to which the distal portion of the pulling shaft 33 is fixed does not need to be fixed to the expansion body 21. In this manner, the distal member 35 can pull the expansion body 21 in a contracting direction. Moreover, when the expansion body 21 is stored in the storage sheath 30, the distal member 35 can be separated from the expansion body 21 to the distal side, so that movement of the expansion body 21 in an extending direction becomes relatively easy to enable the storage capability to be improved.

The operation unit 23 can include a housing 40 to be gripped by an operator, an operation dial 41 that can be rotationally operated by the operator, and a conversion mechanism 42 that is operated in conjunction with the rotation of the operation dial 41. The pulling shaft 33 is held by the conversion mechanism 42 in the inside of the operation unit 23. The conversion mechanism 42 can move the pulling shaft 33 that is held by the conversion mechanism 42 forward and rearward along the axis direction in conjunction with the rotation of the operation dial 41. The conversion mechanism 42 can be, for example, a rack and pinion mechanism.

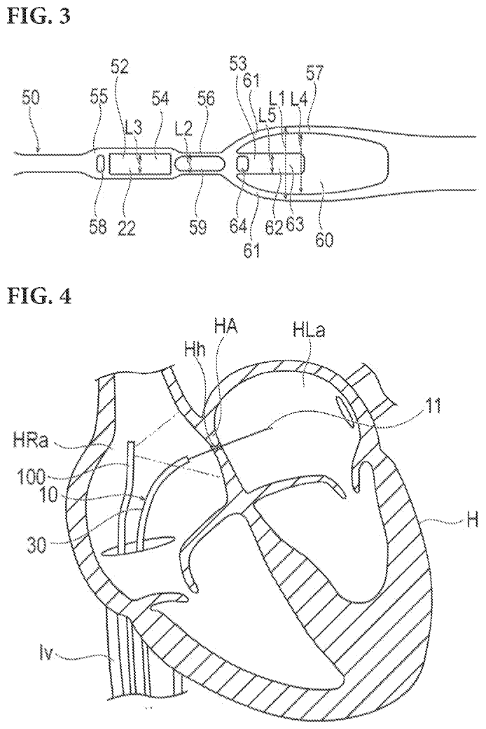

The expansion body 21 will be described in more details. As shown in FIGS. 2 and 3, the expansion body 21 can include a plurality of wire portions 50 in a circumferential direction. In the present embodiment, the four wire portions 50 are provided in the circumferential direction. Note that, the number of the wire portions 50 is not specially limited. The wire portions 50 can respectively expand and contract in a radial direction of the expansion body 21. In a natural state where no external force acts, the expansion body 21 becomes in a reference form in which the expansion body 21 is developed in the radial direction. A proximal portion of the wire portion 50 extends from a distal portion of the outer shaft 31 to the distal side. A distal portion of the wire portion 50 extends from a proximal portion of the distal member 35 to the proximal side. The wire portion 50 is inclined to increase in the radial direction from both end portions to a central portion in an axis direction (axial direction) of the expansion body 21. Moreover, the wire portion 50 can include a clamping portion 51 having a valley shape in an axial central portion, in the radial direction of the expansion body 21.

The clamping portion 51 can include a proximal side clamping portion 52 and a distal side clamping portion 53. The clamping portion 51 further includes a proximal side outer projection portion 55, an inner projection portion 56, and a distal side outer projection portion 57. An interval between the proximal side clamping portion 52 and the distal side clamping portion 53 is preferably opened slightly larger in the axis direction (axial direction) on an outer side than on an inner side in the radial direction, in the reference form, which helps make it relatively easy to dispose a biological tissue between the proximal side clamping portion 52 and the distal side clamping portion 53, from the outer side in the radial direction.

The proximal side outer projection portion 55 is positioned on a proximal side of the proximal side clamping portion 52, and is formed in a convex shape toward the outer side in the radial direction.

The inner projection portion 56 is positioned between the proximal side clamping portion 52 and the distal side clamping portion 53, and is formed in a convex shape toward the inner side in the radial direction. A central through-hole 59 is formed in the inner projection portion 56 such that the inner projection portion 56 bends rather easily.

The distal side outer projection portion 57 is positioned on a distal side of the distal side clamping portion 53, and is formed in a convex shape to the outer side in the radial direction. In the wire portion 50, one distal side through-hole 60 is formed in the vicinity of the distal side outer projection portion 57 and the distal side clamping portion 53. The distal side through-hole 60 penetrates into the radial direction of the expansion body 21. In this manner, the distal side outer projection portion 57 has low flexural rigidity. Therefore, the distal side outer projection portion 57 rather easily deforms to have a convex shape toward the outer side in the radial direction, and easily deforms such that the convex shape becomes flat. Note that, the number of the distal side through-holes 60 is not specially limited. Accordingly, the number of the distal side through-holes 60 may be two or more.

The proximal side clamping portion 52 includes a projection portion 54 that protrudes toward the distal side. The energy transfer element 22 is disposed in the projection portion 54.

The distal side clamping portion 53 can include two outer peripheral portions 61 that are provided on both outer sides in a width direction, and a back support portion 62 that is provided between the two outer peripheral portions 61. The width direction is a direction orthogonal to the axis direction (axial direction) of the expansion body 21, and a direction orthogonal to the radial direction of the expansion body 21. The back support portion 62 includes a receiving surface 63 that can face the energy transfer element 22 that is disposed in the distal side clamping portion 53 when the expansion body 21 expands.

Each of the outer peripheral portions 61 can have a circular arc shape that projects toward the outer side in the width direction. Therefore, between the two outer peripheral portions 61, a relatively wide region in which the back support portion 62 and the distal side through-hole 60 are disposed can be secured. In addition, an outer side of the outer peripheral portion 61 in the width direction becomes relatively smooth, so that the outer peripheral portion 61 can be prevented from being caught on an inner surface of the storage sheath 30, which stores the expansion body 21 inside the storage sheath 30. A maximum width L1 between the outer peripheral portions 61 in the width direction is greater than a maximum width L2 of the inner projection portion 56 in the width direction. Therefore, while maintaining the easiness of bending of the inner projection portion 56, the outer peripheral portions 61 can be formed in a shape expanding in the width direction. Moreover, a maximum width L5 of the receiving surface 63 in the width direction is not specially limited, but is preferably the same as or slightly larger (or greater) than a maximum width L3 of the energy transfer element 22 in the width direction. In this manner, the receiving surface 63 can appropriately receive a biological tissue that is pressed by the energy transfer element 22. Moreover, the maximum width L1 between the outer peripheral portions 61 in the width direction is not specially limited, but is preferably larger (or greater) than the maximum width L3 of the energy transfer element 22 in the width direction. Moreover, a maximum width L4 of the distal side through-hole 60 in the width direction is not specially limited, but is preferably larger (or greater) than the maximum width L3 of the energy transfer element 22 in the width direction.

The back support portion 62 protrudes between the two outer peripheral portions 61 from a site on a side of the inner projection portion 56 of the distal side clamping portion 53 toward a side of the distal side outer projection portion 57. The back support portion 62 is disposed between the two outer peripheral portions 61 to be spaced from the two outer peripheral portions 61. An end portion of the back support portion 62 on the side of the distal side outer projection portion 57 is a free end (i.e., not connected to other portions). Accordingly, the back support portion 62 has a cantilever beam-like form in which a proximal portion of the back support portion 62 is fixed, and warps (i.e., bends) relatively easily. Therefore, the back support portion 62 can relatively easily warp (i.e., bend) than each of the outer peripheral portions 61 due to a force toward the distal side that is received by the receiving surface 63. The back support portion 62 is disposed so as to be clamped by the two outer peripheral portions 61, but does not necessarily need to be strictly positioned in the space positioned between the two outer peripheral portions 61. The back support portion 62 may be disposed so as to be clamped by the two outer peripheral portions 61 at a position slightly shifted from the space positioned between the two outer peripheral portions 61. At least a part of the back support portion 62 is preferably disposed in the space positioned between the two outer peripheral portions 61.

In the back support portion 62, one back support through-hole 64 is formed on the proximal side, in other words, on a side close to the inner projection portion 56. The back support through-hole 64 penetrates into the radial direction of the expansion body 21 (the thickness direction of the wire portions 50). In this manner, the back support portion 62 has low flexural rigidity on a side close to the inner projection portion 56. Therefore, the back support portion 62 can rather easily warp (i.e., bend) due to a force that is received by the receiving surface 63. The number of the back support through-holes 64 is not specially limited. Accordingly, the number of the back support through-holes 64 may be two or more. Moreover, no back support through-hole 64 may be formed in the back support portion 62.

In the present embodiment, the energy transfer element 22 is provided in the proximal side clamping portion 52 and the back support portion 62 is provided in the distal side clamping portion 53, and alternatively, the energy transfer element 22 may be provided in the distal side clamping portion 53 and the back support portion 62 may be provided in the proximal side clamping portion 52.

The wire portion 50 forming the expansion body 21 can have a flat plate shape cut from a cylinder, for example. The wire forming the expansion body 21 can have, for example, a thickness of 50 μm to 500 μm and a width of 0.3 mm to 2.0 mm. However, the wire forming the expansion body 21 has a size outside this range. Moreover, the shape of the wire portion 50 is not limited, and may have a circular shape in a cross section or other shapes in a cross section, for example.

The energy transfer element 22 is provided in the projection portion 54 of the proximal side clamping portion 52, so that when the clamping portion 51 clamps the atrial septum HA, the energy from the energy transfer element 22 is transferred to the atrial septum HA from the right atrium side. In a case where the energy transfer element 22 is provided in the distal side clamping portion 53, the energy from the energy transfer element 22 is transferred to the atrial septum HA from the left atrium side.

The energy transfer element 22 can be configured to include, for example, a bipolar electrode that receives electric energy from an energy supply device serving as an external device. In this case, electricity is supplied among the energy transfer elements 22 disposed in the respective wire portions 50. The energy transfer element 22 and the energy supply device are connected to each other by a conductive wire coated with an insulating coating material. The conductive wire extends to the proximal side of the medical device and outside of the medical device via the shaft portion 20 and the operation unit 23, and is connected to the energy supply device.

Alternatively, the energy transfer element 22 may be configured as a monopolar electrode. In this case, the electricity is supplied between the energy transfer element 22 and a counter electrode plate prepared outside the body. Moreover, the energy transfer element 22 may be a heating element (electrode chip) that generates heat by receiving high-frequency electric energy from the energy supply device. In this case, electricity is supplied among the energy transfer elements 22 disposed in the respective wire portions 50. In addition, the energy transfer element 22 can be configured to include an element that can apply energy to the through-hole Hh, such as an element that provides heating or cooling operation by using microwave energy, ultrasound energy, coherent light such as laser, a heated fluid, a cooled fluid, or a chemical medium, an element that generates frictional heat, or a heater including an electric wire, and a specific form of the energy transfer element 22 is not specially limited.

The wire portion 50 can be formed of a metal material. As the metal material, for example, a titanium-based (Ti—Ni, Ti—Pd, Ti—Nb—Sn, or the like) alloy, a copper-based alloy, stainless steel, β titanium steel, or a Co—Cr alloy can be used. In accordance with an embodiment, an alloy having a spring property such as a nickel titanium alloy may be more preferably used. However, a material for the wire portion 50 is not limited to the materials disclosed, and the wire portion 50 may be formed of other materials.

The shaft portion 20 can include an inner shaft 32 in the inside of the outer shaft 31, and the pulling shaft 33 is stored in an inside of the inner shaft 32. A guide wire lumen is formed in the pulling shaft 33 and the distal member 35 along the axis direction, and a guide wire 11 can be inserted through the guide wire lumen.

The storage sheath 30, the outer shaft 31, the inner shaft 32 of the shaft portion 20 are preferably formed of a material having a certain degree of flexibility. Examples of the materials of the storage sheath 30, the outer shaft 31, and the inner shaft 32 of the shaft portion 20 can include polyolefin such as polyethylene, polypropylene, polybutene, an ethylene-propylene copolymer, an ethylene-vinyl acetate copolymer, an ionomer, or a mixture of two or more of polyolefin such as polyethylene, polypropylene, polybutene, an ethylene-propylene copolymer, an ethylene-vinyl acetate copolymer, and an ionomer, soft polyvinyl chloride resin, polyamide, polyamide elastomer, polyester, polyester elastomer, polyurethane, fluorine resin such as polytetrafluoroethylene, polyimide, PEEK, silicone rubber, and latex rubber.

The pulling shaft 33 can be formed of, for example, an elongated wire material including a super elasticity alloy such as a nickel-titanium alloy and a copper-zinc alloy, a metal material such as stainless steel, and a resin material having comparatively high rigidity. Moreover, the pulling shaft 33 may be formed of the abovementioned wire material coated with a resin material such as polyvinyl chloride, polyethylene, polypropylene, an ethylene-propylene copolymer, or fluorine resin.

The distal member 35 can be formed of, for example, a super elasticity alloy such as a nickel-titanium alloy and a copper-zinc alloy, a metal material such as stainless steel, and a resin material having comparatively high rigidity.

Next, a treatment method using the medical device 10 according to the present embodiment will be described. The treatment method is performed on a patient suffering from a heart failure (left heart failure). More specifically, as shown in FIG. 4, the treatment method is performed on the patient suffering from chronic heart failure, who has a high blood pressure in a left atrium HLa due to myocardial hypertrophy appearing in a left ventricle of the heart H and increased stiffness (hardness).

When the through-hole Hh is formed, an operator delivers an introducer in which a guiding sheath and a dilator are combined with each other to the vicinity of the atrial septum HA. The introducer can be delivered to a right atrium HRa via an inferior vena cava Iv, for example. The introducer can be delivered using the guide wire 11. The operator can insert the guide wire 11 into the dilator, and can deliver the introducer along the guide wire 11. The insertion of the introducer, the insertion of the guide wire 11, and the like to a living body can be performed by using a method such as using an introducer for blood vessel introduction.

Next, the operator causes a puncture device and the dilator to penetrate from the right atrium HRa side toward the left atrium HLa side, thereby forming the through-hole Hh. As for the puncture device, for example, a device such as a wire having a sharp distal end can be used. The puncture device is inserted into the dilator, and is delivered to the atrial septum HA. The puncture device can be delivered to the atrial septum HA instead of the guide wire 11, for example, after the guide wire 11 is extracted from the dilator.

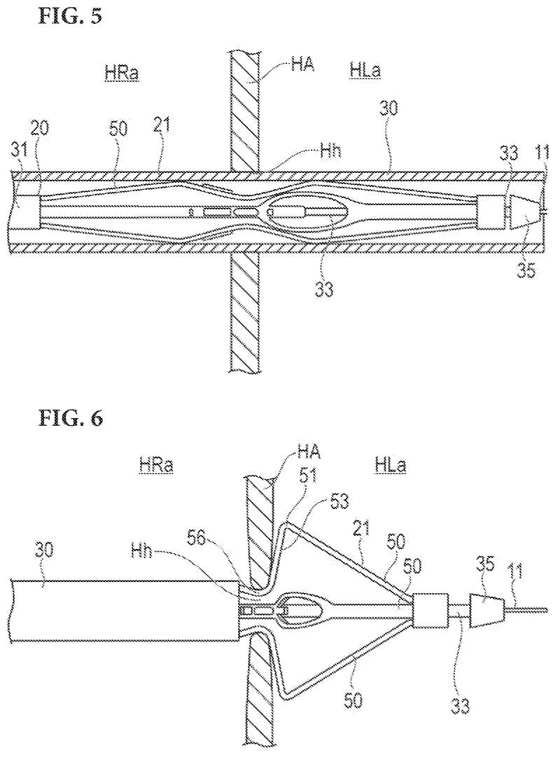

Next, as shown in FIG. 4, the operator delivers the medical device 10 to the vicinity of the atrial septum HA along the guide wire 11 inserted in advance into the left atrium HLa from the right atrium HRa via the through-hole Hh. Further, as shown in FIG. 5, a part of a distal portion of the medical device 10 passes through the through-hole Hh opened in the atrial septum HA, and reaches the left atrium HLa. When the medical device 10 is inserted, the expansion body 21 is in a contracted form in which the expansion body 21 is stored in the storage sheath 30. In the contracted form, the proximal side outer projection portion 55, the inner projection portion 56, and the distal side outer projection portion 57 having convex shapes in a natural state deforms into a shape close to a relatively flat shape, so that the expansion body 21 contracts in the radial direction.

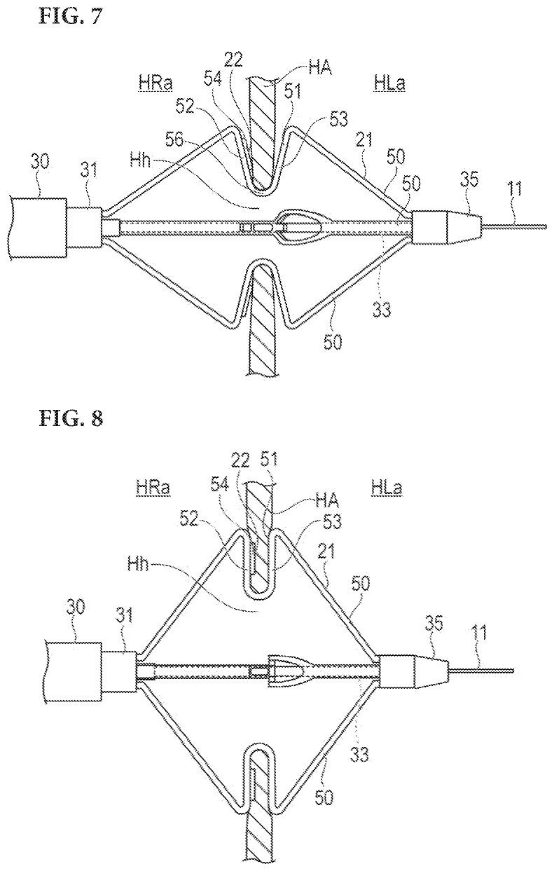

Next, as shown in FIG. 6, the storage sheath 30 is moved to the proximal side, thereby exposing a portion on a distal side of the expansion body 21 in the left atrium HLa. In this manner, the portion on the distal side in the expansion body 21 is developed in the radial direction in the left atrium HLa due to a self-restoring force. Next, as shown in FIG. 7, the storage sheath 30 is moved to the proximal side, thereby exposing the entire expansion body 21. In this manner, the portion on the proximal side in the expansion body 21 is developed in the radial direction in the right atrium HRa due to a self-restoring force. In this process, the inner projection portion 56 is disposed to an inner side of the through-hole Hh. In this manner, the entire expansion body 21 is developed due to the self-restoring force, and is recovered to an original reference form or a form close to the reference form. In this case, the atrial septum HA is disposed between the proximal side clamping portion 52 and the distal side clamping portion 53. In a clamping direction of the biological tissue, the atrial septum HA is disposed between the energy transfer element 22 and the back support portion 62. The expansion body 21 is brought into contact with the through-hole Hh, and thus is not completely returned to the reference form but may be returned to a shape close to the reference form. In this state, the expansion body 21 is not covered with the storage sheath 30, and does not receive a force from the pulling shaft 33. This form of the expansion body 21 can be defined as being included in the reference form (i.e., close to the original form in a natural state where no external force acts on the expansion body 21).

Each of the outer peripheral portions 61 has a circular arc shape that projects to the outer side in the width direction. Therefore, it can be difficult for the outer peripheral portion 61 to be caught on the inner surface of the storage sheath 30. Accordingly, the expansion body 21 including the outer peripheral portions 61 can be smoothly released from the storage sheath 30.

Next, the operator operates the operation unit 23 in a state where the clamping portion 51 holds the atrial septum HA, thereby moving the pulling shaft 33 to the proximal side. In this manner, as shown in FIG. 8, the expansion body 21 that receives a contracting force in the axis direction becomes in an expanded form in which the expansion body 21 has expanded more in the radial direction than in the reference form. The expansion body 21 becomes in the expanded form, so that the proximal side clamping portion 52 and the distal side clamping portion 53 come closer to each other, and the atrial septum HA is clamped between the proximal side clamping portion 52 and the distal side clamping portion 53. In this process, the energy transfer element 22 and the back support portion 62 face each other. The clamping portion 51 in the state of clamping the atrial septum HA further expands to widen the through-hole Hh in the radial direction.

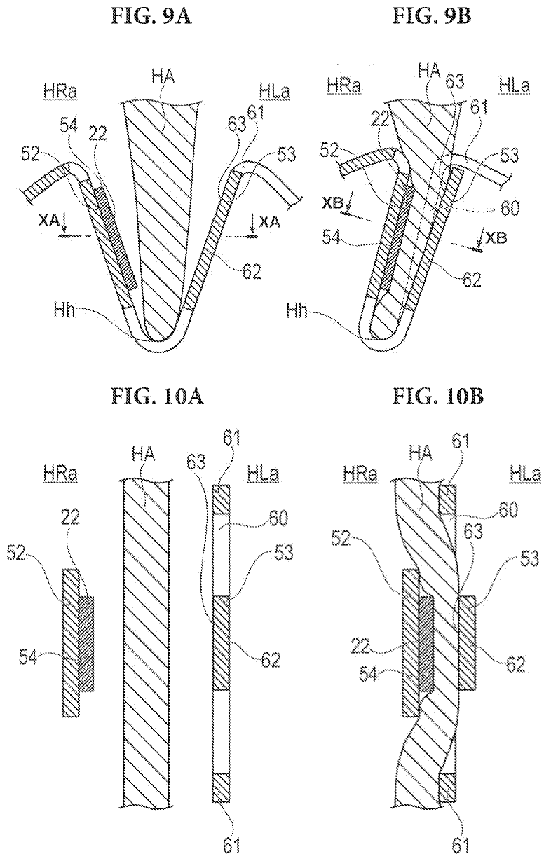

As shown in FIG. 9A and FIG. 10A, when the proximal side clamping portion 52 and the distal side clamping portion 53 come closer to each other from the state where the proximal side clamping portion 52 and the distal side clamping portion 53 are separated from each other, as shown in FIG. 9B and FIG. 10B, the atrial septum HA is clamped between the proximal side clamping portion 52 and the distal side clamping portion 53. Further, the energy transfer element 22 presses the atrial septum HA to the distal side. In this process, the distal side clamping portion 53 causes the back support portion 62 to warp (i.e., bend) to the distal side between the two outer peripheral portions 61, and receives the atrial septum HA that is pressed by the energy transfer element 22, between the two outer peripheral portions 61. The receiving surface 63 of the back support portion 62 receives a force via the atrial septum HA from the energy transfer element 22, and warps (bends) so as to be approximately parallel to the energy transfer element 22. Further, the back support portion 62 causes, while flexibly warping (bending), a repulsion force in a reverse direction of a press-in direction of the energy transfer element 22 to act on the atrial septum HA that is pressed by the energy transfer element 22. In this manner, the energy transfer element 22 comes into close contact with the atrial septum HA. In addition, the energy transfer element 22 can be prevented from locally floating from the atrial septum HA.

In addition, when the energy transfer element 22 is brought into contact with the atrial septum HA, the two outer peripheral portions 61 that sandwich the back support portion 62 between the two outer peripheral portions 61 effectively guide the energy transfer element 22 to the back support portion 62 that is positioned between the outer peripheral portions 61. In this manner, the energy transfer element 22 can press the atrial septum HA that is supported by the two outer peripheral portions 61 while coming into contact with the atrial septum HA, and can press the atrial septum HA against the back support portion 62 that is disposed between the two outer peripheral portions 61. Therefore, the energy transfer element 22 comes into close contact with the atrial septum HA and is relatively difficult to float (or move) from the atrial septum HA, and a position of the energy transfer element 22 relative to the atrial septum HA is stably maintained between the two outer peripheral portions 61.

The atrial septum HA is clamped between the energy transfer element 22 and the back support portion 62, while being clamped in an uneven structure of the projection portion 54 and the distal side through-hole 60. In this process, the back support portion 62 warps (bends), so that the atrial septum HA is rather easily clamped in the uneven structure of the projection portion 54 and the distal side through-hole 60. In this manner, the proximal side clamping portion 52 and the distal side clamping portion 53 support with each other in a circumferential direction of the expansion body 21, so that a position shift of the expansion body 21 in the circumferential direction can be suppressed between the proximal side clamping portion 52 and the distal side clamping portion 53. Therefore, an expanding force of the expansion body 21 can be reliably transferred to the biological tissue. The proximal side clamping portion 52 and the distal side clamping portion 53 clamp the biological tissue when the expansion body 21 expands. When the expansion body 21 expands indicates any one of the middle of the expansion of the expansion body 21, the instant when the expansion body 21 completely expands, and the time from when the expansion body 21 completely expands to when the expansion body 21 contracts.

Further, the maximum width L1 between the outer peripheral portions 61 in the width direction is greater than the maximum width L2 of the inner projection portion 56 in the width direction. Therefore, while flexibly maintaining the inner projection portion 56, a region in which the back support portion 62 is disposed is rather easily secured between the two outer peripheral portions 61. Moreover, the maximum width L1 between the outer peripheral portions 61 in the width direction is greater than the maximum width L3 of the energy transfer element 22 in the width direction. Therefore, the outer peripheral portions 61 rather easily guide the press direction of the energy transfer element 22 towards the back support portion 62. Moreover, the maximum width L4 of the distal side through-hole 60 in the width direction is greater than the maximum width L3 of the energy transfer element 22 in the width direction. Therefore, the outer peripheral portions 61 rather easily guide the press direction of the energy transfer element 22 towards the back support portion 62. In addition, the energy transfer element 22 can easily enter between the outer peripheral portions 61, so that a force in which the proximal side clamping portion 52 including the energy transfer element 22 and the distal side clamping portion 53 including the outer peripheral portions 61 support with each other in the circumferential direction of the expansion body 21 easily acts between the proximal side clamping portion 52 and the distal side clamping portion 53.

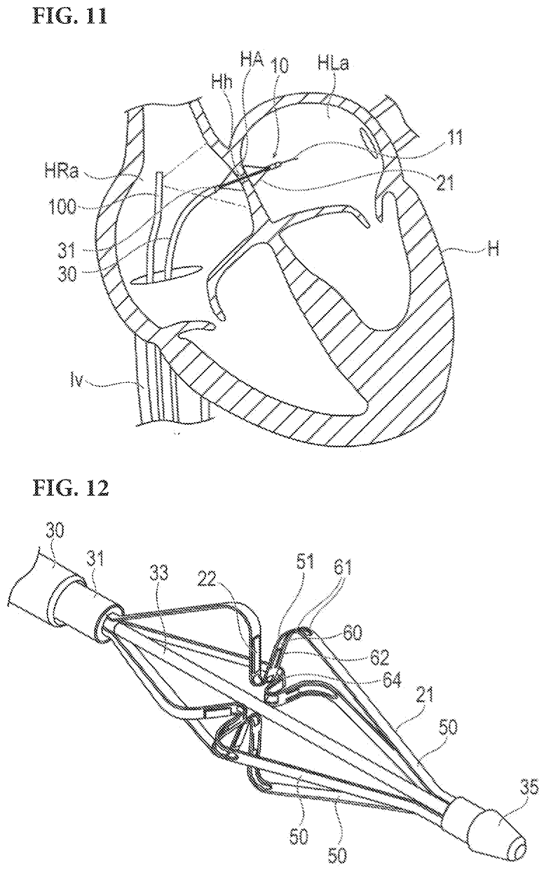

After the through-hole Hh has been enlarged, the hemodynamics is confirmed. As shown in FIG. 11, the operator delivers a hemodynamics confirming device 100 to the right atrium HRa via the inferior vena cava Iv. As for the hemodynamics confirming device 100, for example, an echo catheter can be used. The operator can cause an echo image acquired by the hemodynamics confirming device 100 to be displayed on a display apparatus such as a display, and can confirm a blood volume passing through the through-hole Hh based on a display result of the echo image.

Next, the operator performs maintenance treatment for maintaining the size of the through-hole Hh. In the maintenance treatment, energy is applied to an edge portion of the through-hole Hh through the energy transfer element 22, thereby cauterizing (heating and cauterizing) the edge portion of the through-hole Hh by using the energy. When the biological tissue in the vicinity of the edge portion of the through-hole Hh is cauterized through the energy transfer element 22, a degenerated portion having the degenerated biological tissue is formed in the vicinity of the edge portion. The biological tissue in the degenerated portion is in a state where elasticity is lost, so that the through-hole Hh can maintain a shape widened by the expansion body 21.

The medical device 10 includes the back support portion 62, so that the energy transfer element 22 comes into close contact with the atrial septum HA. Therefore, variations in the degree of cauterization by the energy transfer element 22 can be reduced. Moreover, the energy transfer element 22 can be prevented from locally floating (or moving) from the atrial septum HA. Therefore, the energy transfer element 22 is prevented from supplying energy into blood and an unintended site, and generation of thrombus formation, tissue damage, and the like can be suppressed.

Moreover, the energy transfer element 22 is disposed in the projection portion 54 of the proximal side clamping portion 52. Therefore, the projection portion 54 is pressed against the atrial septum HA, whereby the maintenance treatment is performed in a state where the energy transfer element 22 is embedded in the biological tissue. In this manner, the energy transfer element 22 is prevented from supplying energy into blood and an unintended site, and generation of thrombus formation, tissue damage, and the like can be suppressed.

After the maintenance treatment, the hemodynamics are confirmed again, and in a case where the volume of blood passing through the through-hole Hh reaches a desired amount, the operator decreases the diameter of the expansion body 21, and stores the expansion body 21 in the storage sheath 30 and then extracts the expansion body 21 from the through-hole Hh. The outer peripheral portion 61 has a circular arc shape that projects to the outer side in the width direction, and thus it is difficult for the outer peripheral portion 61 to be caught on the inner surface of the storage sheath 30. Accordingly, the expansion body 21 including the outer peripheral portions 61 is smoothly stored in the storage sheath 30. In addition, the operator extracts the entire medical device 10 to the outside of the living body, and ends the treatment.

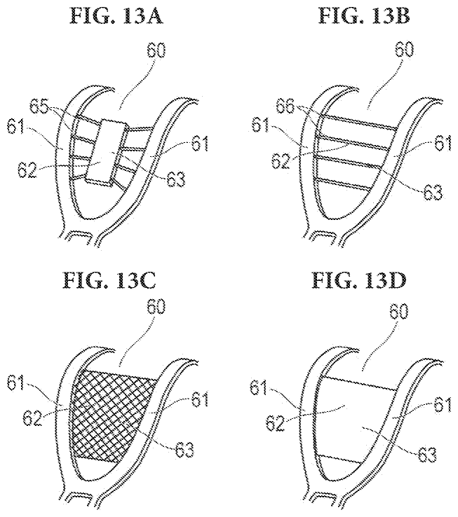

As in the foregoing, the medical device 10 according to the abovementioned embodiment includes: the elongated shaft portion 20, and the expansion body 21 that is provided in a distal portion of the shaft portion 20 and can expand and contract in a radial direction, in which: the expansion body 21 includes a plurality of wire portions 50 that are linked with the shaft portion 30, and at least one clamping portion 51 that is formed by at least one wire portion 50; the clamping portion 51 includes the energy transfer element 22 that outputs energy, and the back support portion 62; the back support portion 62 includes the receiving surface 63 that can face the energy transfer element 22 when the expansion body 21 expands; and the receiving surface 63 can be inclined so as to be approximately parallel to the energy transfer element 22 when the energy transfer element 22 moves toward the back support portion 62. In this manner, in the medical device 10, the receiving surface 63 is approximately parallel to the energy transfer element 22 in accordance with the movement of the energy transfer element 22, so that the energy transfer element 22 can come into close contact with the biological tissue between the energy transfer element 22 and the receiving surface 63. Therefore, variations in the degree of cauterization by the energy transfer element 22 can be reduced. Moreover, the energy transfer element 22 can be prevented from locally floating from the biological tissue. Therefore, the energy transfer element 22 can be prevented from supplying energy into blood and an unintended site, and generation of thrombus formation, tissue damage, and the like due to the cauterization can be suppressed. In the present embodiment, the back support portion 62 is inclined by warping (or bending) due to a force in the axis direction. Note that, the back support portion 62 can be inclined without warping (or bending) by being supported by another deformable member, as shown in a second modification example (see FIG. 13A), which is described later, for example.

Moreover, at least one clamping portion 51 may include the two outer peripheral portions 61 on both sides in a width direction that is a direction orthogonal to the axis direction of the expansion body 21, and a direction orthogonal to the radial direction of the expansion body 21, relative to the back support portion 62. In this manner, the outer peripheral portions 61 effectively guide the energy transfer element 22 that moves toward the back support portion 62 to the back support portion 62 that is positioned between the outer peripheral portions 61. Therefore, the energy transfer element 22 can press the biological tissue supported by the two outer peripheral portions 61, and press the biological tissue against the back support portion 62 that is disposed between the two outer peripheral portions 61. Therefore, the energy transfer element 22 comes into close contact with the biological tissue and can be difficult to float (or move) from the biological tissue, and the position of the energy transfer element relative to the biological tissue can be stably maintained by the two outer peripheral portions 61.

In addition, the two outer peripheral portions 61 each have a convex shape to the outer side in the width direction. In this manner, between the two outer peripheral portions 61, a relatively wide region in which the back support portion 62 is disposed can be secured. In addition, the two outer peripheral portions 61 in the width direction can support the biological tissue in the relatively wide region, so that the energy transfer element 22 and the receiving surface 63 that clamp the biological tissue between the two outer peripheral portions 61 are rather easily maintained in the suitable positions.

In addition, the two outer peripheral portions 61 each have a circular arc shape that smoothly projects to the outer side in the width direction. In this manner, the outer peripheral portion 61 can be stored without being caught on the inner surface of, for example, the storage sheath 30 for storing the expansion body 21 so as to be releasable. Accordingly, the outer peripheral portions 61 can be smoothly stored in the storage sheath 30, and can be smoothly released from the storage sheath 30.

In addition, the maximum width L1 between the outer peripheral portions 61 that sandwich the back support portion 62 between the outer peripheral portions 61 in the width direction is greater than the maximum width L3 of the energy transfer element 22 in the width direction. In this manner, the outer peripheral portions 61 rather easily guide the press direction of the energy transfer element 22 toward the back support portion 62.

In addition, the back support portion 62 moves more than the two outer peripheral portions 61 due to a force in the axis direction to be received from the energy transfer element 22. In this manner, the back support portion 62 can flexibly receive the biological tissue that is pressed by the energy transfer element 22 while moving retracting more than the outer peripheral portion 61 in the press direction of the energy transfer element 22. Therefore, the energy transfer element 22 comes into close contact with the biological tissue, and it is difficult to float (or move) the energy transfer element 22 from the biological tissue.

In addition, the expansion body 21 includes the inner projection portion 56 that projects to the inner side in the radial direction, between the energy transfer element 22 and the back support portion 62, and the maximum width L1 between the outer peripheral portions 61 that sandwich the back support portion 62 between the outer peripheral portions 61 in the width direction is greater than the maximum width L2 of the inner projection portion 56 in the width direction. In this manner, while maintaining the flexibility of the inner projection portion 56, a structure in which the width between the two outer peripheral portions 61 is widened can be obtained. The inner projection portion 56 is flexible to enable the expansion body 21 to be stored in the storage sheath 30, for example. In addition, the inner projection portion 56 is flexible to make it easy to bring the energy transfer element 22 and the back support portion 62 that sandwich the inner projection portion 56 between the energy transfer element 22 and the back support portion 62 such that the energy transfer element 22 and the back support portion 62 can be moved close to or separate from each other.

In addition, the back support portion 62 can have a cantilever beam shape that extends from the wire portion 50. In this manner, the back support portion 62 can flexibly warp (bend) by receiving a force.

In addition, this disclosure further provides a treatment method. The treatment method is a cauterization method of cauterizing a biological tissue, in which: a cauterization device is inserted into a right atrium, the cauterization device including the elongated shaft portion 20, and the expansion body 21 that is provided in a distal portion of the shaft portion 20 and can expand and contract in a radial direction, in which the expansion body 21 includes a plurality of wire portions 50 that are linked with the shaft portion 20, and at least one clamping portion 51 that is formed by at least one wire portion 50, the clamping portion 51 includes the energy transfer element 22 that outputs energy, and the back support portion 62, and the back support portion 62 includes the receiving surface 63 that can face the energy transfer element 22 when the expansion body 21 expands; a distal portion of the expansion body 21 contracted in the radial direction is inserted into the left atrium via the through-hole Hh opened in the atrial septum HA; the distal portion of the contracted expansion body 21 is developed in the left atrium due to a self-restoring force, and a proximal portion of the contracted expansion body 21 is developed in the right atrium HRa due to a self-restoring force, whereby a biological tissue in the vicinity of the edge portion of the through-hole Hh in the atrial septum HA is disposed between the energy transfer element 22 and the back support portion 62; the developed expansion body 21 is expanded in the radial direction, whereby the through-hole Hh is widened while clamping the biological tissue by the clamping portion 51; with the expansion of the through-hole Hh, the biological tissue is pressed toward the back support portion 62 by the energy transfer element 22; the receiving surface 63 of the back support portion 62 is caused to warp so as to be approximately parallel to the energy transfer element 22; a repulsion force from the receiving surface 63 of the back support portion 62 is caused to act on the biological tissue, whereby the energy transfer element 22 is brought into close contact with the biological tissue; and the biological tissue is cauterized by the energy to be output from the energy transfer element 22 having brought into close contact with the biological tissue.

In the cauterization method configured as the above, in accordance with the movement of the energy transfer element 22, the receiving surface 63 becomes approximately parallel to the energy transfer element 22, so that the energy transfer element 22 can be brought into close contact with the biological tissue between the energy transfer element 22 and the receiving surface 63. Therefore, variations in the degree of cauterization by the energy transfer element 22 can be reduced. In addition, the energy transfer element 22 can be prevented from locally floating (or moving) from the biological tissue. Therefore, the energy transfer element 22 can be prevented from supplying energy into blood and an unintended site, and generation of thrombus formation, tissue damage, and the like due to the cauterization can be suppressed.

Note that, this disclosure is not limited to the above-described embodiment, but various changes by those skilled in the art can be made within the technical scope of the present disclosure. For example, as a first modification example shown in FIG. 12, the outer peripheral portion 61 does not need to be formed in a convex shape to the outer side in the width direction.

In addition, the form of the back support portion 62 is not limited. For example, as the second modification example shown in FIG. 13A, the back support portion 62 does not need to have a cantilever beam-like form, but may be supported, for example, by at least one, preferably a plurality of flexible support wires 65 that extend from the two outer peripheral portions 61. The support wire 65 is thinner and more flexible than the outer peripheral portion 61. A constituent material for the support wire 65 is not specially limited as long as it can flexibly deform, and for example, a super elasticity alloy such as a nickel-titanium alloy and a copper-zinc alloy, a metal material such as stainless steel, polyolefin such as polyethylene, polypropylene, polybutene, an ethylene-propylene copolymer, an ethylene-vinyl acetate copolymer, an ionomer, or a mixture of two or more of materials disclosed above, soft polyvinyl chloride resin, polyamide, polyamide elastomer, polyester, polyester elastomer, polyurethane, fluorine resin such as polytetrafluoroethylene, and a resin material such as polyimide, PEEK, silicone rubber, or latex rubber can be preferably used. The back support portion 62 is linked with the outer peripheral portions 61 only by the support wires 65. Therefore, the back support portion 62 can be inclined relatively easier by receiving a force in the axis direction (axial direction), than the outer peripheral portion 61. A plurality of the support wires 65 may be provided and disposed approximately in parallel to each other. In addition, a plurality of the support wires 65 may be provided and disposed in a mesh shape.

Moreover, as a third modification example shown in FIG. 13B, the back support portion 62 may include at least one, preferably a plurality of back support wires 66 having both ends that are fixed to the outer peripheral portions 61. A constituent material for the back support wire 66 is not specially limited as long as it can flexibly deform, and for example, the above-mentioned material applicable to the support wire 65 can be used suitably. The receiving surface 63 of the back support portion 62 is formed by the plurality of the back support wires 66 with gaps.

Moreover, as a fourth modification example shown in FIG. 13C, the back support portion 62 may be a mesh-like member that is disposed between the two outer peripheral portions 61. The mesh-like member may include, for example, a plurality of fine lines, or a flexible member having a large number of holes being formed in the flexible member. A constituent material for the mesh-like member is not specially limited as long as it can flexibly deform, and for example, the above-mentioned material applicable to the support wire 65 can be used suitably.

Moreover, as a fifth modification example shown in FIG. 13D, the back support portion 62 may be an elastically deformable film body that is disposed between the two outer peripheral portions 61. As a constituent material for the film body, for example, resin such as fluorine resin, polyethylene, and polypropylene, thermoplastic elastomer, rubber, or the like can be used suitably.

The detailed description above describes embodiments of a medical device for applying energy to a biological tissue. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an elongated shaft portion; and
an expansion body that is provided in a distal portion of the shaft portion, the expansion body configured to expand and contract in a radial direction, wherein
the expansion body includes a plurality of wire portions that are linked with the shaft portion, and at least one clamping portion that is formed by at least one of the wire portions,
the clamping portion includes an energy transfer element configured to output energy, and a back support portion,
the back support portion includes a receiving surface that is configured to face the energy transfer element when the expansion body expands, and
the receiving surface is configured to be inclined to be approximately parallel to the energy transfer element, when the energy transfer element moves toward the back support portion.

2. The medical device according to claim 1, wherein the at least one clamping portion includes two outer peripheral portions on both sides in a width direction that is a direction orthogonal to an axial direction of the expansion body, and a direction orthogonal to the radial direction of the expansion body, relative to the back support portion.

3. The medical device according to claim 2, wherein the two outer peripheral portions each have a convex shape to an outer side in the width direction.

4. The medical device according to claim 3, wherein the two outer peripheral portions each have a circular arc shape that smoothly projects to the outer side in the width direction.

5. The medical device according to claim 2, wherein a maximum width between the two outer peripheral portions that sandwich the back support portion between the two outer peripheral portions in the width direction is larger than a maximum width of the energy transfer element in the width direction.

6. The medical device according to claim 2, wherein the back support portion moves larger than the two outer peripheral portions due to a force in the axis direction to be received from the energy transfer element.

7. The medical device according to claim 2, wherein
the expansion body includes an inner projection portion that projects to an inner side in the radial direction between the energy transfer element and the back support portion; and
the maximum width between the two outer peripheral portions that sandwich the back support portion between the two outer peripheral portions in the width direction is larger than a maximum width of the inner projection portion in the width direction.

8. The medical device according to claim 1, wherein the back support portion has a cantilever beam shape that extends from the wire portion.

9. The medical device according to claim 2, wherein the back support portion is a member that is supported by at least one flexible support wire that extends from the two outer peripheral portions that sandwich the back support portion between the two outer peripheral portions.

10. The medical device according to claim 2, wherein the back support portion is at least one flexible back support wire that extends from the two outer peripheral portions that sandwich the back support portion between the two outer peripheral portions.

11. The medical device according to claim 2, wherein the back support portion is a mesh-like member that extends from the two outer peripheral portions that sandwich the back support portion between the two outer peripheral portions.

12. The medical device according to claim 2, wherein the back support portion is a film body that extends from the two outer peripheral portions that sandwich the back support portion between the two outer peripheral portions.

13. A medical device comprising:
an elongated shaft portion; and
an expansion body that is provided in a distal portion of the shaft portion, and is configured to expand and contract in a radial direction, wherein
the expansion body includes a plurality of wire portions that are linked with the shaft portion, and at least one clamping portion that is formed by at least one of the wire portions,
the clamping portion includes an energy transfer element configured to output energy, and a back support portion,
the at least one clamping portion includes two outer peripheral portions on both sides in a width direction,
the back support portion includes at least one flexible back support wire that extends from the two outer peripheral portions that sandwich the back support portion between the two outer peripheral portions so as to form a receiving surface, and
the receiving surface is configured to face the energy transfer element when the expansion body expands and be inclined to be approximately parallel to the energy transfer element, when the expansion body expands and the energy transfer element moves toward the back support portion.

14. The medical device according to claim 13, wherein the width direction is a direction orthogonal to an axial direction of the expansion body, and a direction orthogonal to the radial direction of the expansion body, relative to the back support portion.

15. The medical device according to claim 13, wherein the at least one flexible back support wire comprises a plurality of back support wires, the plurality of back support having both ends fixed to the two outer peripheral portions.

16. The medical device according to claim 15, wherein the receiving surface of the back support portion is formed by the plurality of the back support wires with gaps.

17. A method for treatment, the method comprising:
expanding a through-hole formed in an atrial septum to allow a right atrium and a left atrium of a heart failure patient to communicate with each other;
confirming hemodynamics of blood flow in a vicinity of the through-hole;
performing maintenance treatment for maintaining a size of the through-hole with a medical device comprising an elongated shaft portion, and an expansion body that is provided in a distal portion of the shaft portion, the expansion body configured to expand and contract in a radial direction, wherein the expansion body includes a plurality of wire portions that are linked with the shaft portion, and at least one clamping portion that is formed by at least one of the wire portions, the clamping portion includes an energy transfer element configured to output energy, and a back support portion, and wherein the back support portion includes a receiving surface;
expanding the expansion body in the through hole so that the receiving surface faces the energy transfer element; and
moving the energy transfer element toward the back support portion so that the receiving surface is approximately parallel to the energy transfer element.

18. The method according to claim 17, further comprising:
applying energy to an edge portion of the through-hole through the energy transfer element to cauterize the edge portion of the through-hole.

19. The method according to claim 17, wherein the at least one clamping portion includes two outer peripheral portions on both sides in a width direction that is a direction orthogonal to an axial direction of the expansion body, and a direction orthogonal to the radial direction of the expansion body, relative to the back support portion.

20. The method according to claim 19, wherein the back support portion is a member that is supported by at least one flexible support wire that extends from the two outer peripheral portions that sandwich the back support portion between the two outer peripheral portions.
